(19) United States
(12) Patent Application Publication
(10) Pub. No.: US 2011/0213214 A1
(43) Pub. Date: Sep. 1, 2011

(54) MECHANICALLY DEPLOYABLE TRACHEAL TUBE SENSOR

(75) Inventors: Alan Finneran, Offaly (IE); Garret Coady, Dublin (IE); John Desmond, Kildare (IE); Mark Cleary, Dublin (IE); David Powell, Dublin (IE); Patrick Dowling, Kildare (IE)

(73) Assignee: Nellcor Puritan Bennett LLC, Boulder, CO (US)

(21) Appl. No.: 12/713,323

(22) Filed: Feb. 26, 2010

(51) Int. Cl. A61B 5/00; A61M 16/04

(52) U.S. Cl. 600/301; 128/207.14

(57) ABSTRACT

Various embodiments of a tracheal tube having a mechanically deployable sensor are provided. Certain embodiments of the tracheal tube may be capable of mechanically deploying the sensor during intubation to sense one or more indicators of blood flow characteristics, such as a level of blood gases and/or blood analytes, in the respiratory tract. The mechanically deployable sensor may be configured to abut the tracheal mucosa of a patient or not contact the tracheal wall at all during deployment. The sensor may be further adapted to remain in a recess disposed in the tracheal tube prior to deployment and exit the recess when acquiring measurements.

### BACKGROUND

The present disclosure relates generally to medical devices and, more particularly, to airway devices, such as tracheal tubes.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

Tracheal tubes are often placed in the airway of a patient in medical situations that necessitate protection of the airway from possible obstruction or occlusion. For instance, tracheal tubes may be used in emergency situations, such as when a patient experiences cardiac or respiratory arrest. The underlying condition that necessitates intubation of the patient may also cause a drop in aortic pressure, leading to low blood flow to non-critical organs, such as the respiratory tract, to compensate for an increased need for blood flow to critical organs, such as the brain. A decrease in blood flow to the respiratory tract may be detected by assessing the level of blood gases and/or blood analytes present in the tracheal mucosa.

Some traditional systems measure the level of blood gases and/or blood analytes in the respiratory tract by introducing a sensor into the trachea and contacting the tracheal mucosa. However, for critically ill patients already intubated with a tracheal tube, introduction of an additional sensing device can be uncomfortable and burdensome. Accordingly, systems that deploy the sensor from the tracheal tube already in place in the respiratory tract have been developed. However, such systems often fall short of expectations since they may compromise one or more of the functions of the tracheal tube, require the sensor to directly contact the mucosa, and so forth. Accordingly, there exists a need for improved systems that measure blood gases and/or blood analytes in the respiratory tract without interrupting the proper functioning of the tracheal tube.

### BRIEF DESCRIPTION OF THE DRAWINGS

Advantages of the disclosed techniques may become apparent upon reading the following detailed description and upon reference to the drawings in which:

FIG. 1 is an elevational view of an exemplary endotracheal tube with a mechanically deployable sensor located above a cuff in accordance with aspects of the present disclosure;

FIG. 2 is an elevational view of an exemplary endotracheal tube with a mechanically deployable sensor located below a cuff in accordance with aspects of the present disclosure;

FIG. 3 illustrates the endotracheal tube of FIG. 1 positioned in a trachea of a patient lying in a semirecumbent position with a deployed sensor in accordance with aspects of the present disclosure; and

FIG. 4 is an elevational view of an exemplary endotracheal tube with a mechanically deployable sensor located between two cuffs in accordance with aspects of the present disclosure.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

As described in detail below, embodiments of an endotracheal tube (ETT) having a mechanically deployable sensor are provided. The mechanically deployable sensor is configured to enter the trachea via a lumen disposed lengthwise along the tracheal tube. To that end, the lumen may be adapted to receive one or more conductors or support cables that facilitate a bidirectional exchange of data, power, and so forth between the sensor and an external support system. The tracheal tube may be disposable rather than reusable, capable of sensing one or more indicators of blood flow characteristics, capable of conveying gas to and from a patient, and capable of deploying one or more sensors during intubation. While a patient is being intubated and/or extubated, the mechanically deployable sensor may be configured to remain in a recess disposed in the tracheal tube. During use, the sensor may be mechanically deployed either manually or automatically. Furthermore, when deployed, the sensor may be adapted to abut the tracheal mucosa of the patient or, when desired not to contact the tracheal wall at all. Nevertheless, the sensor is configured to measure a presence or amount of at least one blood gas and/or blood analyte, such as carbon dioxide, oxygen, or pH, in the trachea during deployment. In this way, embodiments of the disclosed ETT may be used to indirectly monitor the cardiac state of a patient by monitoring the level of blood gases and/or blood analytes in the respiratory tract. That is, measurements of such gas and analyte levels in the trachea may be used to determine parameters relating to cardiac output, such as blood flow, and may provide insight into possible cardiac pathologies, such as perfusion failure.

The devices and techniques provided herein may minimize the complexity of the system used to sense blood gases and/or blood analytes in a patient airway as compared to traditional designs because the sensing system is integral with the main tubular body of the tracheal tube. That is, additional assemblies need not be attached to the tracheal tubes to enable sensing capabilities; these capabilities are inherent in the design and manufacture of the tracheal tubes. As such, the provided devices and systems may enable the ability to sense blood flow parameters in intubated patients without introducing a bulky or cumbersome sensing device in additional to the tracheal tube. That is, the mechanically deployable sensor is integrated into the tracheal tube, thus obviating the need for another device to be introduced into the trachea. The foregoing feature may have the effect of increasing patient comfort as compared to traditional sensing systems.

Embodiments of the present invention may include deployment of the sensor in a variety of locations along the length of the tracheal tube. For instance, the sensor may be deployable to a position located above a sealing cuff, a position located below a sealing cuff, or a position located between two cuffs. Additionally, the sensor may be deployed in an optimized location such that interference from accumulated mucus or secretions is minimized. The foregoing features may have the effect of maintaining the functionalities of traditional tracheal tubes (e.g., providing an unobstructed airway path) while endowing the tracheal tubes with new functionalities (e.g., measuring blood gases and/or analytes).

It should be noted that the provided tracheal tubes and methods of operating the tracheal tubes may be used in conjunction with auxiliary devices, such as airway accessories, ventilators, humidifiers, and so forth, which may cooperate with the tracheal tubes to maintain airflow to and from the lungs of the patient. For instance, the tracheal tubes may be placed in the trachea and coupled to a ventilator to protect the airway from possible obstruction or occlusion in emergency situations, such as when a patient experiences cardiac or respiratory arrest. For further example, the tracheal tubes may be coupled to an interface circuit and/or a monitor that is configured to receive data from the sensor, process such data, and display the processed data to an end user (e.g., medical technician, doctor, nurse, etc.).

Furthermore, although the embodiments of the present invention illustrated and described herein are discussed in the context of endotracheal tubes, it should be noted that presently contemplated embodiments may include a mechanically deployable sensor coupled to any of a variety of suitable airway devices. For example, the mechanically deployable sensor may be coupled to a tracheostomy tube, a Broncho-Cath™ tube, a specialty tube, or any other airway device. Indeed, any device designed for use in an airway of a patient may be coupled to the mechanically deployable sensor. Furthermore, as used herein, the term “tracheal tube” may include an endotracheal tube, a tracheostomy tube, a Broncho-Cath™ tube, a specialty tube, or any other airway device.

Turning now to the drawings, FIG. 1 is an elevational view of an exemplary ETT 10 in accordance with aspects of the present disclosure. The endotracheal tube 10 includes a central tubular body 12 with proximal and distal ends 14 and 16, respectively. In the illustrated embodiment, the proximal end 14 is outfitted with a connector 18 that may be attached to a mechanical ventilator during operation. The distal end 16 terminates in an opening 20 and may be placed in a trachea of a patient during operation to maintain airflow to and from the lungs. A Murphy's eye 22 may be located on the tubular body 12 opposite the opening 20 to prevent airway occlusion when the tube assembly 10 is improperly placed within the trachea.

As illustrated, a cuff 24 that may be inflated to seal against the walls of a body cavity (e.g., the trachea) may be attached to the distal end 16 of the tubular body 12. The cuff 24 may be inflated via an inflation lumen 26 terminating in an inflation tube 28 connected to a fixture 30 located at the proximal end 14 of the tubular body 12. A first shoulder 32 and a second shoulder 34 of the cuff 24 secure the cuff 24 to the tubular body 12. In some embodiments, the first shoulder 32 and/or the second shoulder 34 may be folded up inside a lower end of the cuff 24.

The tubular body 12 and the cuff 24 may be formed from materials having desirable mechanical properties (e.g., puncture resistance, pin hole resistance, tensile strength, and so forth) and desirable chemical properties (e.g., biocompatibility). In one embodiment, the walls of the cuff 24 may be made of a polyurethane (e.g., Dow Pellethane® 2363-80A) having suitable mechanical and chemical properties. In other embodiments, the walls of the cuff 24 may be made of a suitable polyvinyl chloride (PVC). In certain embodiments, the cuff 24 may be generally sized and shaped as a high volume, low pressure cuff that may be designed to be inflated to pressures between about 15 cm and 30 cm of water. The cuff 24 may be any of a variety of suitable cuffs, such as a tapered cuff or a non-tapered cuff.

A sensor 36 mounted on a tip of a deployment member 38 (e.g., polymeric tube, plastic support, etc.) is configured to be mechanically deployed in the trachea during use. To that end, a lumen 40 is disposed along the tubular body 12 of the tracheal tube 10 from the proximal end 14 to a location above the cuff 24. The deployment member 38 is adapted to be partially disposed in the lumen 40 during intubation. That is, while the patient is being intubated or extubated, the sensor 36 is configured to rest in a recess 42 disposed in the tubular body 12, and a first portion 44 of the deployment member 38 is adapted to terminate outside of the lumen 40. After intubation, the deployment member 38 may be slid lengthwise along the tubular body 12, thus deploying the sensor 36. The sensor 36 may be deployed to a position that abuts the tracheal wall or is in close proximity to the tracheal mucosa. Furthermore, in some embodiments, the recess 42 may include a ramp portion that is adapted to guide the sensor 36 toward the tracheal wall as the sensor 36 is deployed. Nevertheless, during deployment, the sensor 36 is configured to measure the presence or level of one or more blood gases and/or blood analytes. After deployment, the sensor 36 may be mechanically withdrawn from its deployment position and returned to the recess 42.

It should be noted that the deployment member 38 may be any of a variety of suitable deployment apparatuses with a variety of functionalities. For instance, the deployment member 38 may be a polymeric tube that is configured to undergo a variety of tensile and compressive forces (e.g., as the deployment member is slid along the length of the tracheal tube). The deployment member 38 may be further adapted to encase one or more conductors that terminate in the sensor 36 or may be coupled to the conductors. For example, the conductors terminating in the sensor 36 may be secured to a support that facilitates the deployment of the sensor 36. To that end, the deployment member 38 may function to encase one or more conductors, to provide the structure necessary to withstand tensile and compressive forces, and to facilitate the deployment of the sensor 36.

In the presently contemplated embodiments, the sensor may contact the tracheal mucosa directly to obtain a blood gas or blood analyte measurement or, alternatively, the sensor may obtain the measurement via equilibration with gases or analytes located in the tracheal cavity adjacent the mucosa and/or the tracheal wall tissue. Accordingly, the sensor may be any suitable carbon dioxide, oxygen, pH, or other gas or analyte sensor, such as an electrochemical sensor, a fluorometric sensor, or a mid-infrared sensor. Furthermore, the sensor may be configured to simultaneously or sequentially measure more than one gas or analyte level.

The deployment member 38 terminates in a connector 46 that may couple the output of the sensor 36 to one or more external devices or systems. For example, in some embodiments, the deployment member 38 may include one or more conductors or support cables that facilitate a bidirectional exchange of data, power, and so forth between the sensor 36 and an external support system. That is, during or after intubation, the sensor 36 may be supplied with power and may export data via the lumen 40. In this way, the lumen 40 and the conductors located in the member 38 facilitate data exchange between the sensor 36 located within the patient and the support system positioned outside the patient. For instance, a presence or level of carbon dioxide may be measured by the deployed sensor 36 within the trachea and transferred in real time to a monitoring device via the conductors disposed in the lumen 40.

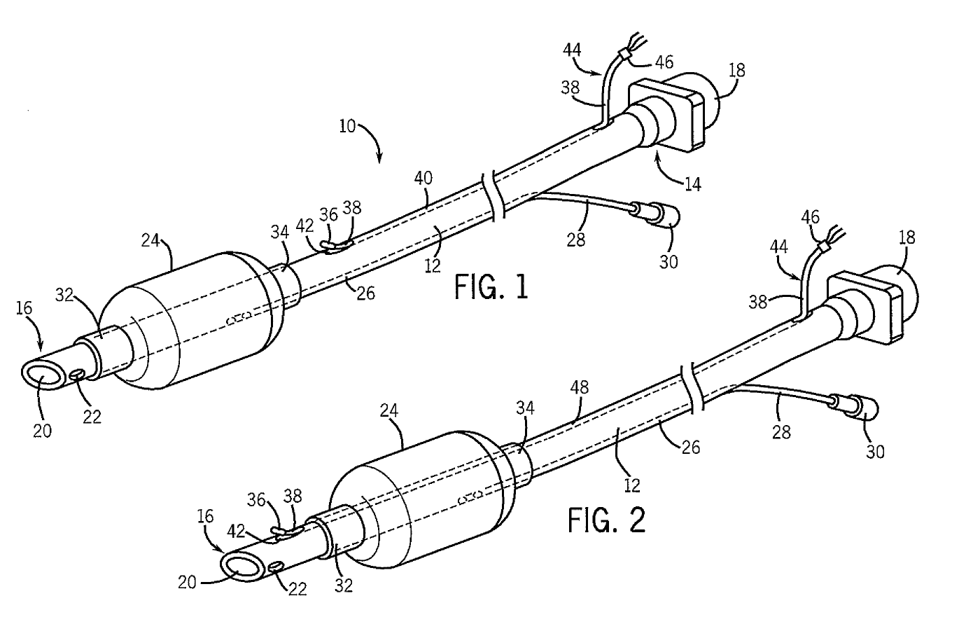

In the embodiment of FIG. 1, the lumen 40, terminating in the recess 42 through which the sensor 36 is deployed, itself terminates above the cuff 24. In contrast, the embodiment of FIG. 2 includes a lumen 48 that extends from the proximal end 14 of the tubular body 12 and terminates in the recess 42 below the cuff 24. That is, in the embodiment of FIG. 2, the sensor 36 is deployable below the cuff 24 toward the distal end 16 of the tubular body 12. As before, the sensor 36 may be configured to deploy to a location proximate to the tracheal wall or may deploy to a position abutting the tracheal mucosa. As such, the sensor 36 may be adapted to measure a presence or level of one or more blood gases and/or blood analytes and to communicate the measured data to an external device or system via one or more conductors located in the deployment member 38.

The embodiments of FIG. 1 and FIG. 2 illustrate a single sensor 36 mounted on the tip of a single deployment member 38. In further embodiments, however, multiple sensors may be mounted on multiple tubes. The sensors may be configured to sense the same parameter (e.g., the same blood gas and/or blood analyte) or different parameters. Furthermore, the sensors may be configured to sense different parameters at the same radial position around the circumference of the tracheal tube or the same parameter at different radial locations around the tracheal tube. For instance, one embodiment may include a carbon dioxide sensor mounted on the tip of a first tube disposed in a first lumen and an oxygen sensor mounted on the tip of a second tube disposed in a second lumen. Each lumen may then be disposed in the wall of the tubular body of the tracheal tube at different radial locations. For further example, another embodiment may include a single sensor mounted on the tip of a deployment member disposed in a single lumen, and the sensor may be adapted to measure the presence or level of more than one blood gas or blood analyte simultaneously or sequentially.

It should be further noted that a variety of acquisition methods may be employed in conjunction with the mechanically deployable sensor to acquire data. For example, the sensor may be configured to remain deployed while the patient is intubated and take measurements at preset time intervals. The sensor may also be configured to deploy, record a measurement, and return to its predeployment position until another measurement is desired. The sensor may be further adapted to remain in a deployed position but to obtain measurements only when directed by an external control system. Finally, the sensor may be adapted to be manually deployed and to acquire data as desired by an operator.

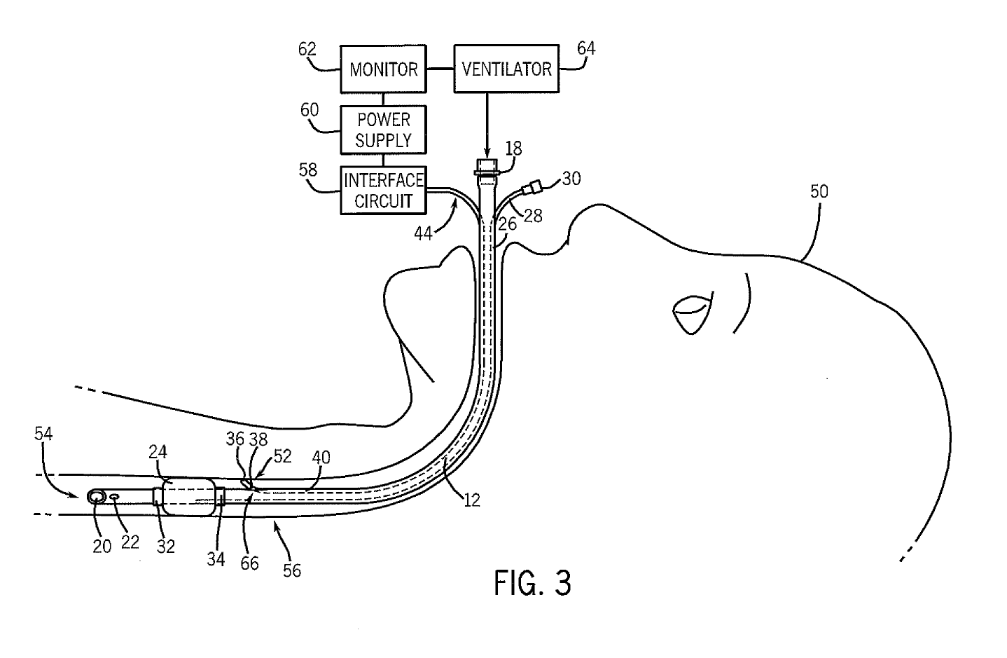

FIG. 3 illustrates an exemplary system including a patient 50 intubated with the endotracheal tube 10 of FIG. 1 in accordance with embodiments of the present invention. As illustrated, the patient 50 is shown lying in a semirecumbent position as may be typical during long term intubations. In the illustrated embodiment, the lumen 40 is positioned within the tubular body 12 such that when the deployment member 38 is disposed in the lumen 40, the sensor 36 is deployable on the side of the cuff 24 that faces the ventral side of the patient during intubation in the semirecumbent position. That is, in the embodiment shown, the sensor 36 may be deployed to contact a first side or wall 52 of a trachea 54 of the patient. In some embodiments, this position may offer advantages over sensors configured to be deployed close to a second side or wall 56 of the trachea 54 since mucus may be prone to accumulating near the dorsal side of the patient during intubation in a semirecumbent position. By deploying the sensor 36 near the ventral side of the patient in these embodiments, interference from accumulated secretions may be prevented and one or more suctioning ports may be included if desired. However, other embodiments may feature sensors 36 that are minimally affected or unaffected by secretion accumulation. In such embodiments, the sensor 36 may be placed in any desirable location around the circumference of the trachea.

The lumen 40 and the deployment member 38 terminating in the connector 46 may couple one or more devices or systems to the sensor 36 during intubation. To this end, the first portion 44 of the deployment member 38 is positioned external to the intubated patient 50 when the patient is lying in the semirecumbent position as in FIG. 3. In the illustrated embodiment, the deployment member 38 is communicatively coupled to an interface circuit 58 that is configured to receive and process measurement data acquired by the sensor 36. The interface circuit 58 is coupled to a power supply 60 that provides power for the sensor 36 and any electronics associated with the sensor 36. The interface circuit 58 may also facilitate the transfer of power to the sensor 36 in some embodiments. The power supply 60 is further coupled to a monitor 62 that is adapted to interpret and display the measurements received from the sensor 36 via the interface circuit 58. To that end, the monitor 62 may include a memory, a display, code configured to provide a specific output, and so forth. For example, the monitor 62 may include software adapted to integrate measurements taken at preset intervals over a predetermined period of time and/or to average or otherwise process measurements taken from multiple positions within the trachea 54. The monitor 62 may be connected to a ventilator 64 that supplies air to the patient 50 through connector 18.

In further embodiments, the deployment member 38 may be coupled to additional devices and systems not shown in FIG. 3. For example, the connector 46 may couple to a motion generator that is adapted to slide the deployment member 38 through the lumen 40 to deploy the sensor 36. As the deployment member 38 is moved through the lumen 40, the sensor 36 may exit recess 42 along a ramp 66 that directs the sensor to the tracheal wall 52. The sensor 36 acquires one or more measurements while deployed near or against the tracheal mucosa. The motion generator may then slide the member 38 back into the lumen 40, returning the sensor 36 back to its predeployment position in the recess 42.

Still further, in other embodiments, the sensor 36 may be adapted to unidirectionally or bidirectionally communicate with one or more external devices via wireless communication. That is, in some embodiments, the sensor 36 may not be coupled to the external devices via the conductors. In such embodiments, the sensor 36 may wirelessly communicate with devices such as a monitor, ventilator, mobile phone, PDA, or central communications point. Further embodiments may feature a single conductor that couples the sensor 36 to the power supply 60, while data communication occurs via a wireless route.

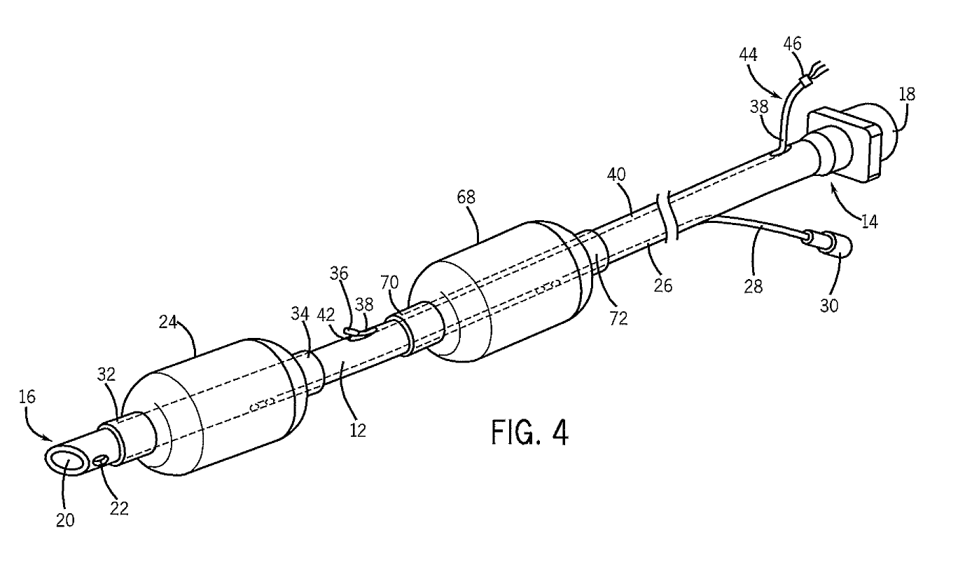

FIG. 4 is an elevational view of another exemplary embodiment of an ETT assembly in accordance with aspects of the present invention. As before, the tracheal tube assembly includes the cuff 24 that is adapted to seal against the walls of the trachea during use. However, in this embodiment, a second cuff 68 is attached to the tubular body 12 above the first cuff 24 toward the proximal end 14 of the tracheal tube. The second cuff 68 is secured to the tubular body via a third shoulder 70 and a fourth shoulder 72. The recess 42 through which the sensor 36 mounted on the tip of the member 38 is configured to deploy is located above the first cuff 24 and below the second cuff 68. As before, the lumen 40 extends from a location toward the proximal end 14 of the tubular body 12 to the recess 42.

During use, the first cuff 24 and the second cuff 68 are configured to be inflated to seal against the walls of the trachea. When inflated, a cavity is formed in the area disposed between cuff 24 and cuff 68. Isolation of the cavity located between the cuffs 24 and 68 may facilitate the acquisition of measurements in embodiments in which the sensor 36 is not configured to contact and/or seal against the wall of the tracheal mucosa. That is, in such embodiments, the blood gases and/or blood analytes may equilibrate between the mucosa and the isolated cavity, thus ensuring that the acquired measurements are indicative only of gas parameters in the mucosa and not end tidal movement in the air. Additionally, the sensor 36 may be deployed against the tracheal wall in the isolated cavity. In such embodiments, the cuff 68 may facilitate the alignment of the tracheal tube when the sensor 36 is deployed.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Rather, the various embodiments may cover all modifications, equivalents, and alternatives falling within the spirit and scope of the disclosure as defined by the following appended claims.

What is claimed is:

1. A tracheal tube, comprising:
a tubular body having an open distal end for ventilating a patient;
a cuff disposed around the tubular body above the open distal end and configured to be inflated to seal the cuff against a wall of a trachea;
a deployment member extending along the tubular body and configured to move within the tubular body; and
a sensor mounted on, at or near a tip of the deployment member and configured to measure a level of a blood gas and/or a blood analyte in the trachea.

2. The tracheal tube of claim 1, comprising a deployment lumen extending from a proximal end of the tubular body to a location above the cuff, and wherein the deployment member is disposed in the deployment lumen.

3. The tracheal tube of claim 1, wherein the sensor is deployable at a location along the tubular body above the cuff.

4. The tracheal tube of claim 1, comprising a deployment lumen extending from a proximal end of the tubular body to a location below the cuff and wherein the deployment member is disposed in the deployment lumen.

5. The tracheal tube of claim 1, wherein the sensor is configured to deploy to a position abutting a tracheal mucosa and/or to a position in close proximity to the tracheal mucosa as the deployment member moves within the tubular body.

6. The tracheal tube of claim 1, comprising a recess in the tubular body adapted to receive the sensor before and/or after deployment.

7. The tracheal tube of claim 6, wherein the recess comprises a ramp portion configured to guide the sensor to a deployment position.

8. The tracheal tube of claim 1, comprising a connector configured to attach to a proximal end of the tubular body to communicatively couple the tracheal tube to a controlled ventilation device.

9. The tracheal tube of claim 1, wherein the deployment member is configured to receive one or more conductors to communicatively couple the sensor to at least one of a monitor, a ventilator, a power supply, or an interface circuit.

10. A tracheal tube, comprising:
a tubular body having an open distal end for ventilating a patient;
a first cuff disposed around the tubular body above the open distal end and configured to be inflated to seal the first cuff against a wall of a trachea;
a second cuff disposed around the tubular body above the first cuff and configured to be inflated to seal the second cuff against the wall of the trachea;
a deployment member configured to slide lengthwise along the tubular body to a cavity formed between the first cuff and the second cuff when inflated; and
a sensor mounted at or near a distal end of the deployment member and configured to deploy in the cavity to measure a level of a blood gas and/or a blood analyte in the trachea.

11. The tracheal tube of claim 10, comprising a deployment lumen extending from a proximal end of the tubular body to a location between the first cuff and the second cuff, and wherein the deployment member is disposed in the deployment lumen.

12. The tracheal tube of claim 10, comprising a recess having a ramp portion configured to guide the sensor to a deployment position.

13. The tracheal tube of claim 10, wherein the deployment member is configured to receive one or more conductors to communicatively couple the sensor to at least one of a monitor, a ventilator, a power supply, or an interface circuit.

14. The tracheal tube of claim 10, comprising an inflation lumen extending along the tubular body between a location on the tracheal tube positioned outside the patient when in use to a location of the first cuff and/or the second cuff positioned inside the patient, wherein the inflation lumen is adapted to deliver inflation gas to the respective cuff.

15. The tracheal tube of claim 10, wherein the blood gas and/or blood analyte is carbon dioxide, oxygen, pH, or a combination thereof.

16. A tracheal tube, comprising:
a tubular body having an open distal end for ventilating a patient;
a cuff disposed around the tubular body above the open distal end and configured to be inflated to seal the cuff against a wall of a trachea; and
a mechanically deployable sensor disposed in a ramped recess in the tubular body and configured to deploy from the recess to measure a level of a blood gas and/or a blood analyte in or near a mucosa of the trachea.

17. The tracheal tube of claim 16, wherein the mechanically deployable sensor is mounted on a tip of a polymeric tube.

18. The tracheal tube of claim 16, comprising a second cuff disposed around the tubular body above the cuff and configured to form a cavity between the cuff and the second cuff when inflated.

19. The tracheal tube of claim 18, wherein the mechanically deployable sensor is configured to deploy in the cavity.

20. The tracheal tube of claim 16, wherein the mechanically deployable sensor is disposed on a tip of a polymeric tube that extends lengthwise within a deployment lumen disposed along the tubular body.
